(19) United States
(12) Patent Application Publication
(10) Pub. No.: US 2007/0010770 A1
(43) Pub. Date: Jan. 11, 2007

(54) Pneumatic liner with pressure relief valve and method of supporting an extremity with a pneumatic liner with pressure relief valve

(76) Inventor: Richard E. Gildersleeve, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/477,893

(22) Filed: Jun. 29, 2006

Related U.S. Application Data
(60) Provisional application No. 60/697,200, filed on Jul. 7, 2005.

(51) Int. Cl. A61F 5/00

(52) U.S. Cl. 602/13

(57) ABSTRACT

The present pneumatic liner includes a pressure relief valve. The valve limits the maximum pressure that can be reached within at least one bladder in the liner. When the threshold pressure is reached, the valve opens to vent excess air. The liner is thus adapted to prevent overinflation, which could restrict circulation in the wearer's limb. Also disclosed is a method of supporting an extremity using the liner.

### CROSS-REFERENCE TO RELATED APPLICATIONS

This application claims priority to provisional application Ser. No. 60/697,200, filed on Jul. 7, 2005, the entire contents of which are hereby expressly incorporated by reference.

### BACKGROUND OF THE INVENTION

1. Field of the Invention

The present invention relates to pneumatic liners for orthopedic devices and methods of using the same.

2. Description of the Related Art

Several orthopedic devices, such as ankle walkers, include pneumatic liners that enhance patient comfort.

### SUMMARY OF THE INVENTION

The preferred embodiments of the present pneumatic liner and method of using the same have several features, no single one of which is solely responsible for their desirable attributes. Without limiting the scope of this pneumatic liner and method as expressed by the claims that follow, its more prominent features will now be discussed briefly. After considering this discussion, and particularly after reading the section entitled “Detailed Description of the Preferred Embodiments,” one will understand how the features of the preferred embodiments provide advantages, which include enabling adjustment of form-fitting characteristics of the liner without the risk of over inflation.

One embodiment of the present pneumatic liner and method comprises a liner for an orthopedic walker. The liner includes a sleeve portion constructed of a relatively soft and flexible fabric, at least one bladder disposed within the sleeve portion, a pump adapted to inflate the at least one bladder, and a pressure relief valve in fluid communication with the bladder. The pressure relief valve is adapted to vent excess air from the at least one bladder once a threshold pressure is reached inside the at least one bladder.

One embodiment of the present pneumatic liner and method comprises a method of supporting a wearer's extremity. The method comprises the steps of placing the extremity within a flexible sleeve, the sleeve including at least one inflatable bladder, securing the sleeve about the extremity, and inflating the bladder until a pressure relief valve operably connected to the bladder opens to vent excess air within the bladder.

### BRIEF DESCRIPTION OF THE DRAWINGS

The preferred embodiments of the present pneumatic liner and method of using the same, illustrating its features, will now be discussed in detail. These embodiments depict the novel and non-obvious pneumatic liner and method shown in the accompanying drawings, which are for illustrative purposes only. These drawings include the following figures, in which like numerals indicate like parts:

FIG. 1 is a front perspective view of one embodiment of a pneumatic liner for an ankle walker that includes a pressure relief valve;

FIG. 2 is a front perspective view of one embodiment of a walker that is adapted to receive the pneumatic liner of FIG. 1;

FIG. 3 is a cross-sectional view of one embodiment of a pressure relief valve that is adapted for use with the pneumatic liner of FIG. 1;

FIG. 4 is a cross-sectional view of another embodiment of a pressure relief valve that is adapted for use with the pneumatic liner of FIG. 1;

FIG. 5 is a cross-sectional view of another embodiment of a pressure relief valve that is adapted for use with the pneumatic liner of FIG. 1;

FIG. 6 is a cross-sectional view of another embodiment of a pressure relief valve that is adapted for use with the pneumatic liner of FIG. 1;

FIG. 7 is a cross-sectional view of another embodiment of a pressure relief valve that is adapted for use with the pneumatic liner of FIG. 1;

FIG. 8 is a cross-sectional view of another embodiment of a pressure relief valve that is adapted for use with the pneumatic liner of FIG. 1;

FIG. 9 is a cross-sectional view of another embodiment of a pressure relief valve that is adapted for use with the pneumatic liner of FIG. 1;

FIG. 10 is a cross-sectional view of another embodiment of a pressure relief valve that is adapted for use with the pneumatic liner of FIG. 1; and

FIG. 11 is a cross-sectional view of another embodiment of a pressure relief valve that is adapted for use with the pneumatic liner of FIG. 1.

### DETAILED DESCRIPTION OF THE PREFERRED EMBODIMENTS

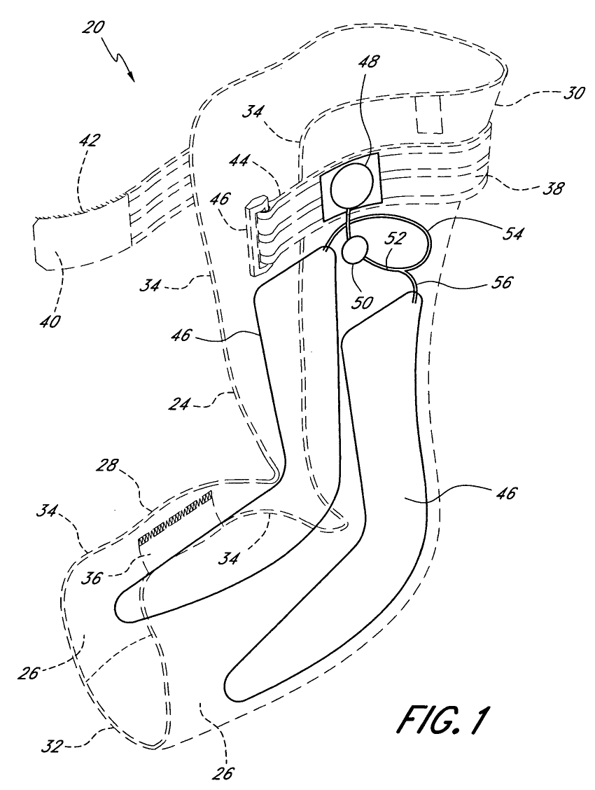

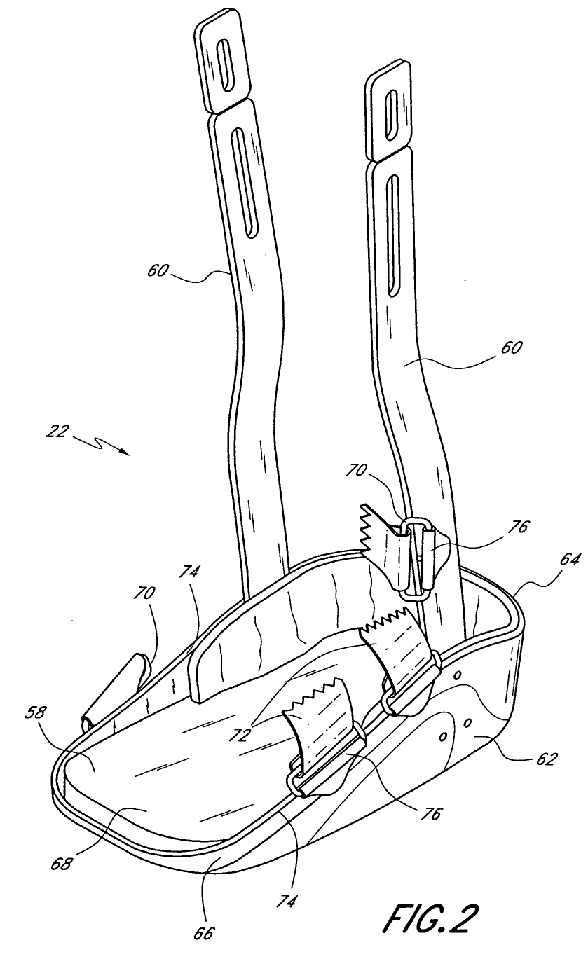

FIG. 1 illustrates a pneumatic liner 20 that is adapted for use in an ankle walker. The liner 20 is adapted to be received within a rigid ankle walker, such as the walker 22 illustrated in FIG. 2. The liner 20 provides a comfortable interface between the wearer's leg, ankle and/or foot and the rigid components of the walker. Application Ser. No. 29/196,058, filed on Dec. 18, 2003 (“the '058 application”), discloses several additional rigid ankle walkers that are adapted for use with the present liner. The '058 application is commonly owned with the present application, and the entirety of the '058 application is expressly incorporated by reference herein.

The illustrated liner 22 includes a sleeve portion 24 that is preferably constructed of a soft and flexible but durable material. The sleeve portion may comprise multiple layers, such as a padding layer (not shown) sandwiched between first and second outer fabric layers 26. In the illustrated embodiment, the sleeve portion 24 is shaped substantially as a boot, and includes a lower foot-receiving portion 28 and an upper ankle/leg-receiving portion 30. The wearer's toes may protrude through a front opening 32 of the lower foot-receiving portion 28, and first and second front edges 34 of the sleeve portion are adapted to overlap one another to envelop the wearer's leg, ankle and foot. The liner 22 may include a closure mechanism (not shown), such as snaps, buttons, hook-and-loop fastener, etc. In the illustrated embodiment, a flap 36 secured to the front edge 34 of one side of the foot-receiving portion 28 includes hook material (not shown) on one side that mates with loop material (not shown) on the front edge 28 of the other side of the foot-receiving portion. The liner 22 may include any number of additional flaps.

Near an upper edge, the ankle/leg-receiving portion 30 may include a strap 38 that extends around the wearer's lower leg when the liner 22 is worn. Ends of the strap 38 extend forward from the front edges 34 of the ankle/leg-receiving portion. In the illustrated embodiment, a first end 40 of the strap includes hook material 42, and a second end 44 of the strap includes a D-ring 46. The first end 40 is adapted to pass through the D-ring 46 and fold back over the strap 38 to mate with a patch of loop material (not shown). The wearer can thus adjust the tightness of the liner 22 by adjusting the tension within the strap 38.

In the illustrated embodiment, the liner 20 includes medial and lateral air chambers 46 that are adapted to be inflated with ambient air using an air bladder pump 48. The bladders 46 may be provided between the first and second outer fabric layers 26 of the liner 20, for example. The inflatable bladders 46 enable the wearer to customize the fit of the liner to his or her own limb. As the bladders 46 inflate, the liner 20 form fits to the wearer's leg, ankle and/or foot.

Overinflation of the bladders 46 can create discomfort for the wearer and/or restrict circulation within the wearer's extremity. Thus, it is desirable to limit the maximum pressure within each bladder 46. The illustrated liner 20 includes a pressure relief valve 50 (shown schematically in FIG. 1) that sets such an upper limit. The valve is preferably configured to remain closed until the pressure within the bladders 46 reaches the maximum upper limit, at which point the valve opens and vents excess air. In the embodiment of FIG. 1, one valve 50 is provided, and it is located along a first portion 52 of an air conduit that comprises a single fluid pathway from the pump 48. Downstream from the pump, the air conduit branches into a second portion 54 and a third portion 56, and each portion 54, 56 leads to its own respective bladder 46. The valve 50 is thus in fluid communication with both of the bladders 46, and able to control overinflation of both. However, those of ordinary skill in the art will appreciate that the liner could include dual pressure relief valves 50, with each valve being located along one of the second and third portions 54, 56 of the air conduit, or with each valve incorporated into a respective one of the bladders 46. In such alternative embodiments, each bladder 46 includes its own pressure relief valve, such that overinflation of each bladder 46 can be separately controlled. In these embodiments, the valves 50 may be configured such that the maximum pressure for one of the bladders 46 is different than the maximum pressure for the other bladder 46. Those of skill in the art will appreciate that although the illustrated liner includes two bladders, the present pressure relief valves may also be employed in liners including any number of bladders, such as one bladder, three bladders, etc.

To support an extremity using the liner 20 of FIG. 1, a wearer would place his or her lower leg, ankle and foot within the liner and secure the liner about his or her extremity. For example, the wearer may pull the front edges 34 toward each other in an overlapping fashion until the liner snugly encases the wearer's foot, ankle and lower leg, and then secure the overlapping front edges. The wearer may then further tighten the ankle/leg-receiving portion 30 by threading the first end 40 of the strap 38 through the D-ring 46, pulling the strap snug and folding the first end back over the strap 38 to secure the first end. Next, the wearer may inflate the bladders 46 by operating the pump 48. The wearer may continue to inflate the bladders until the maximum pressure is reached, at which point the pressure relief valve 50 opens and vents excess air.

The walker 22 illustrated in FIG. 2 includes a foot bed 58 for receiving the wearer's foot (encased in a liner), and first and second rigid uprights 60. An outer shell 62 of the foot bed is generally constructed of a rigid and durable material that is preferably lightweight. For example, glass-reinforced nylon may be used. The rigid uprights 60 are similarly generally constructed of a durable material that is preferably lightweight. For example, aluminum alloys, such as 6061-T6 or 5052-H32 may be used. A heel cup 64 at a posterior end of the foot bed receives the wearer's heel, while an anterior end 66 of the foot bed receives the wearer's toes. An upper surface 68 of the foot bed may comprise a resilient padding material, such as polyurethane foam. The walker 22 may include a plurality of D-rings 70 that are adapted to receive straps 72. In the illustrated embodiment, the D-rings 70 are pivotably attached to side walls 74 of the foot bed 58 and to the rigid uprights 60 via flexible strap tabs 76. Four D-rings 70 are illustrated in FIG. 2, however, those of ordinary skill in the art will appreciate that any number of fewer or additional D-rings may be provided. The D-rings provide anchoring points for the straps 72 that secure the rigid portions of the walker to the wearer's leg, ankle and foot.

FIGS. 3-11 illustrate examples of pressure relief valves that are adapted for use with the liner 20 of FIG. 1. Those of skill in the art will appreciate that the illustrated valves are merely examples, and that other valve designs could be incorporated with the liner 20 to achieve similar benefits. With reference to FIG. 3, one embodiment of the present pressure relief valve 78 includes an opening 80 in a sealing surface 82. The sealing surface may be, for example, a wall of an air conduit 52, 54, 56, or a wall of a bladder 46. A sealing member 84 covers the opening and provides an airtight seal. Edges of the opening extend upward and present flat sealing surfaces 86 that mate with the sealing member. A cover 88 surrounds the sealing member 84 and the opening 80. A spring 90 extends between an inner surface of the cover 88 and the sealing member 84, forcing the sealing member against the opening. The cover is preferably constructed of a material that is rigid enough to provide a support surface against which the spring 90 can bear without significantly deforming the cover 88. For example, metals and rigid plastics may be used. The spring force provided by the spring is preferably set at a level that allows the bladders 46 to inflate to a predetermined pressure. Once the pressure is reached, the force of the air pushing the sealing member 84 away from the opening 80 overcomes the force provided by the spring 90, and the valve 30 opens. The air escapes to the ambient through a hole 92 in the cover. In the illustrated embodiment, one hole is provided. However, those of skill in the art will appreciate that more holes could be provided. In fact, in each of the embodiments described herein, any number of holes may be provided as desired.

In FIG. 3, the sealing member 84 is substantially disk-shaped, and the cover 88 is substantially cylindrical, with a flat upper surface. The pressure relief valve 94 illustrated in FIG. 4 is similar to the pressure relief valve 78 illustrated in FIG. 3. However, the sealing member 96 is shaped substantially as a cylinder with one open end, and the cover 98 has a domed upper surface 100. Further, the edges of the opening 102 extend upward to present tapered seal points 104 that mate with the sealing member.

In FIG. 5, the pressure relief valve 106 includes a dome-shaped cover 108, and a substantially dome-shaped sealing member 110. A lower edge 112 of the sealing member engages an upwardly extending side edge 114 of the opening 116 to create the seal. A spring 118 maintains the sealing member in its sealing configuration. Bosses 120 on the upper surface of the sealing member 110 and the lower surface of the cover 108 set a maximum range of movement for the sealing member. Preferably, the bosses prevent the lower edge 112 of the sealing member from disengaging the side edge 114 of the opening. The sealing member 110 thus remains centered over the opening 116, even when the valve is venting excess air. In the illustrated embodiment, the cover 108 includes two holes 122 that allow excess air to escape. Those of skill in the art will appreciate that fewer or more holes could be provided.

The pressure relief valve 124 illustrated in FIG. 6 is substantially similar to that of FIG. 5. However, in FIG. 6 the cover 126 is indented at its top surface 128, and the indentation provides the spring force that keeps the sealing member 130 in place. The cover 126 is preferably made of a resilient material that holds the illustrated shape until the pressure in the bladders 46 crosses the predetermined threshold, at which point the cover deforms to allow the sealing member 130 to move away from the opening 132. When the excess air escapes, the force of the cover 126 tending to return to its original shape pushes the sealing member 130 back down over the opening 132. The sealing member is shaped as a cylinder that is open at both ends, with a central disk portion 134. Bosses 136 on the inner surface of the cover mate with the upper end of the sealing member and hold it in place over the opening. In the illustrated embodiment, two holes 138 are provided for venting excess air.

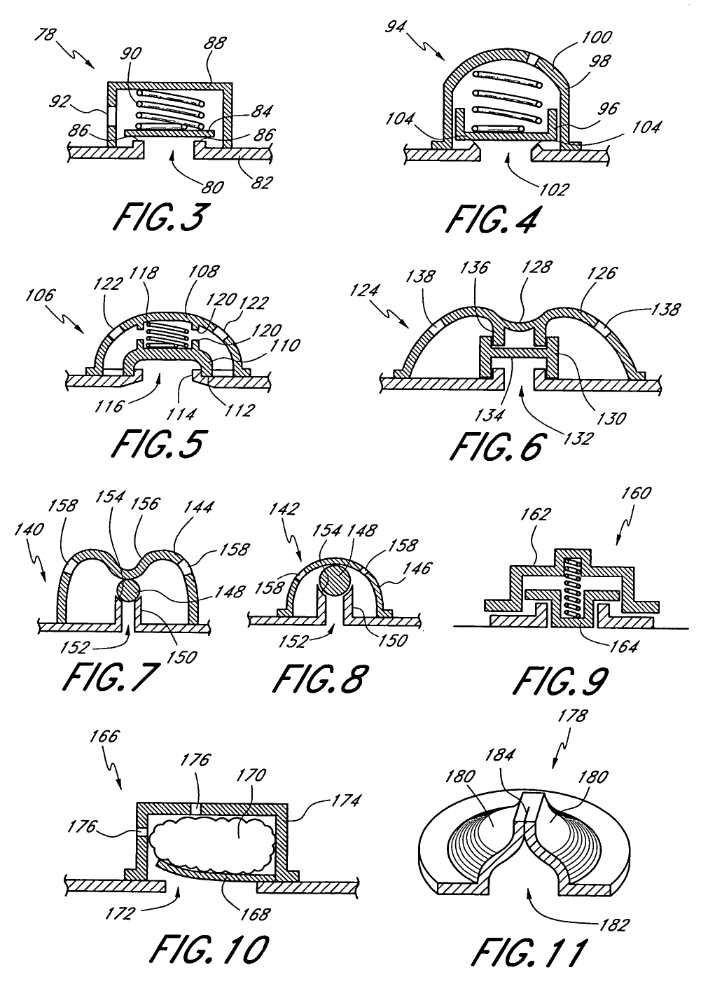

The valves 140, 142 of FIGS. 7 and 8 are substantially identical, except for the shapes of the covers 144, 146. In each, a spherical sealing member 148 rests atop an upwardly extending edge 150 of the opening 152. The upper surface 154 of each edge 150 is preferably contoured to mate with the sealing member 148 and seal the opening 152. The sealing member may be, for example, made of stainless steel. In the embodiment of FIG. 7, the cover 144 is substantially cylindrical, with an indented upper surface 156. In the embodiment of FIG. 8, the cover 146 is substantially dome-shaped. Each cover 144, 146 provides the spring force that maintains the sealing member 148 in the sealing position until the threshold pressure is reached within the bladders 46. Each cover includes two holes 158 for venting excess air.

The valve 160 of FIG. 9 is substantially similar to the embodiments of FIGS. 3 and 4, except for the shapes of the cover 162 and sealing member 164, as illustrated. The valve 166 of FIG. 10 is substantially similar to the embodiment of FIG. 3, except that the spring force holding the sealing member 168 in place is provided by a compressible material 170, such as foam or felt. The sealing member 168 is preferably resilient, such that when the pressure within the bladders 46 reaches the threshold, the sealing member deforms as shown in FIG. 10 to vent the excess air. After the pressure drops below the threshold, the compressible material 170 forces the sealing member 168 back down over the opening 172. In the illustrated embodiment, the cover 174 includes two holes 176 for venting the excess air.

The valve 178 of FIG. 11 comprises a duckbill valve. Side walls 180 of the opening 182 taper toward one another and meet at a slit 184. Abutting surfaces of the slit 184 seal the opening. The material comprising the side walls 180 preferably has sufficient rigidity to maintain the opening in a sealed configuration until the threshold pressure is reached within the bladders 46. When the threshold pressure is reached, the air forces the side walls of the duckbill valve 178 apart, allowing the excess air to escape. After the pressure drops below the threshold, the side walls come together again at the slit to reseal the opening.

In the embodiments described above, the spring force holding the sealing member over the opening is provided by coil springs and resilient materials. Those of skill in the art will appreciate that other types of springs could be used instead, such as Bellville springs and elastomers. Those of skill in the art will further appreciate that the sealing members illustrated above could be constructed of any appropriate material, such as metals, plastics and elastomeric materials. Those of skill in the art will further appreciate that the configurations of the various valve components are merely examples. Alternative configurations are encompassed by the claims below. Further, the pressure relief valves described above are well adapted for use in orthopedic devices other than walkers. Any orthopedic device including an inflatable bladder could include a pressure relief valve as described herein. The walker liner illustrated is merely an example.

### Scope of the Invention

The above presents a description of the best mode contemplated for carrying out the present pneumatic liner with pressure relief valve, and of the manner and process of making and using it, in such full, clear, concise, and exact terms as to enable any person skilled in the art to which it pertains to make and use this pneumatic liner. This pneumatic liner is, however, susceptible to modifications and alternate constructions from that discussed above that are fully equivalent. Consequently, this pneumatic liner is not limited to the particular embodiments disclosed. On the contrary, this pneumatic liner covers all modifications and alternate constructions coming within the spirit and scope of the pneumatic liner as generally expressed by the following claims, which particularly point out and distinctly claim the subject matter of the pneumatic liner.

What is claimed is:

1. A liner for an orthopedic bracing device, comprising:
a sleeve portion constructed of a relatively soft and flexible fabric;
at least one bladder secured to the sleeve portion;
a pump secured to the sleeve portion and adapted to inflate the at least one bladder; and
a pressure relief valve in fluid communication with the bladder;
wherein the pressure relief valve is adapted to vent excess air from the at least one bladder once a threshold pressure is reached inside the at least one bladder.

2. The liner of claim 1, wherein the at least one bladder is disposed between first and second layers of the sleeve.

3. The liner of claim 2, wherein the first and second layers are constructed of fabric.

4. The liner of claim 1, wherein the pressure relief valve comprises a duckbill valve.

5. The liner of claim 1, wherein the pressure relief valve comprises a sealing member, a spring member and a cover.

6. The liner of claim 5, wherein the sealing member is adapted to selectively close an opening in a sealing surface of the liner.

7. The liner of claim 6, wherein the spring member biases the sealing member toward the opening.

8. The liner of claim 7, wherein a first end of the spring member bears against the cover and a second end of the spring member bears against the sealing member.

9. The liner of claim 5, wherein the spring member is one of a coil spring or a resilient material.

10. The liner of claim 1, wherein the pressure relief valve comprises a sealing member and a cover.

11. The liner of claim 9, wherein the sealing member is adapted to selectively close an opening in a sealing surface of the liner.

12. The liner of claim 11, wherein the cover is constructed of a resilient material.

13. The liner of claim 12, wherein the cover provides a spring force that biases the sealing member toward the opening.

14. The liner of claim 1 in combination with an ankle walker, the walker comprising a foot bed portion and first and second rigid uprights.

15. A method of supporting a wearer's extremity, comprising the steps of:
placing the extremity within a flexible sleeve, the sleeve including at least one inflatable bladder;
securing the sleeve about the extremity; and
inflating the bladder until a pressure relief valve operably connected to the bladder opens to vent excess air within the bladder.

16. The method of claim 15, further comprising the step of placing the sleeve-wrapped extremity within an orthopedic bracing device.

17. The method of claim 16, further comprising the step of securing the bracing device about the sleeve-wrapped extremity.

18. The method of claim 17, wherein the orthopedic bracing device comprises an ankle walker.

19. The method of claim 18, wherein the step of securing the bracing device about the sleeve-wrapped extremity comprises wrapping at least one strap around at least a portion of the sleeve-wrapped extremity.
